(19) United States
(12) Patent Application Publication
(10) Pub. No.: US 2003/0161922 A1
(43) Pub. Date: Aug. 28, 2003

(54) Method of treating and preparing food in a cooking device, control panel for a cooking device and cooking device comprising a control panel

(76) Inventor: Johannes Hillmann, Neu-Ulm (DE)

Correspondence Address: JANSSON, SHUPE & MUNGER, LTD, 245 MAIN STREET, RACINE, WI, 53403, US

(21) Appl. No.: 10/373,227

(22) Filed: Feb. 24, 2003

(30) Foreign Application Priority Data
Feb. 25, 2002 (EP) 02 004 106.7

(51) Int. Cl. G01N 33/02

(52) U.S. Cl. 426/523; 426/231

(57) ABSTRACT

The present invention refers to a method of treating and preparing food in a cooking device, in which a plurality of cooking parameters can be set, wherein the cooking process is carried out by means of an adjustable cooking program, wherein the setting of the cooking parameters is carried out by an input device. By means of the input device a point in an at least two-dimensional map is controlled by the operator of the cooking device, in which at least two cooking parameters are defined, and the adjusted point is implemented into the cooking program as part of the cooking process. The present invention also comprises a control panel for a cooking device, in particular for a steam cooking device and a cooking device equipped with this control panel.

### FIELD OF THE INVENTION

The present invention refers to a method of treating and preparing food in a cooking device.

The present invention further refers to a control panel for a cooking device, as well as a cooking device comprising such a cooking panel, in particular an improved steam cooking device.

### BACKGROUND OF THE INVENTION

Methods of treating and preparing food by means of various cooking devices have been known for many years. In carrying out the cooking process, one must select from a number of different parameters. Conventionally, a selection is made from one of the operating modes offered by a cooking device that is dependent on the food to be cooked. Operating modes of this type are, for instance, hot air, steaming, frying, regenerating, etc.

After the mode of operation has been selected, a selection from other parameters takes place which correspond to this mode of operation. In addition to the selected mode of operation, these selections must be input by the person operating the cooking device, such as the temperature, humidity, etc., and these values must be input independent of one another in successive steps.

The process of cooking food is usually carried out by way of one or more programed steps, e.g. heating-up, steaming, roasting and subsequent browning, wherein each individual step must be selected by the operator who then inputs the respective parameters.

The input is conventionally made on a display panel having a line-shaped structure that shows the corresponding choices in parameters as a plurality of lines and allowing each parameter to be controlled, e.g. by means of a rotary knob. When reaching the correct value, the parameter is selected by pressing a certain confirmation key for this program step.

### SUMMARY OF THE INVENTION

The object of the present invention is to provide a method of treating and preparing food in a cooking device where the parameters of the cooking process can be selected in an extremely simple manner.

The object of the invention is further to provide a control panel for a cooking device, in particular a steam cooking device, by means of which the determination of the cooking process can be managed in a very simple manner.

The present invention is furthermore based on the objective of providing a cooking device in which each of the above-mentioned aims are also realized.

According to the present invention, a method of treating and preparing food in a cooking device is provided where a plurality of cooking parameters can be selected by an operator of the cooking device and where the cooking process can be carried out by means of an adjustable cooking program. The method comprises the steps of the operator choosing a point on an at least two-dimensional map by means of an input device wherein at least two cooking parameters are selected on the cooking device and then implementing that selection into the cooking program as part of the cooking process.

Thereby it is advantageously achieved that the parameter settings selected by the operator, usually the cook, can be made in not only a simple but also quick manner. Moreover, the operating safety is clearly increased. Therefore input errors are avoided to a great extent.

One preferred embodiment of this method is where the temperature or humidity to be reached in the cooking chamber, the velocity of the fan wheel of the cooking device, the browning degree of the food to be cooked and/or the core temperature of the food to be cooked can be selected as cooking parameters.

In order to provide the operator (cook) and in particular unskilled or semi-skilled persons with aid when selecting the cooking parameters, panels for different cooking processes are predefined and are displayed on the map so that one can be selected by the input device. These predefined panels are displayed either graphically or photographically and are regarded by an expert as the “language of cooking”.

In an especially advantageous manner, every point on the map can be reached so that any possible combination of preselected cooking parameters can be set. This may for instance be the temperature and the humidity in the cooking chamber, thus setting the cooking chamber climate. As an alternative, different parameters, such as cooking time, core temperature, fan wheel velocity, browning degree of the food to be cooked, can form the basis of the map and can be selected by the operator, depending on the step of the cooking process. This allows the operator to select from an entire spectrum of possible cooking parameters, with extreme settings also being possible.

The treatment and preparation of food is known to be very stressful as personnel must perform a huge number of different jobs in any type of kitchen, no matter whether it is a canteen kitchen or a gourmet restaurant. The simpler and safer a cooking process can be made, the better. The food shall also be protected against over-cooking to achieve its maximum edible quality.

In an especially advantageous manner, control over the program point on the map is implemented by a multi-dimensional input device, such as a rocker switch, a joystick, a computer mouse or a touch pad, the latter being known from a laptop.

The rocker switch may, for instance, be designed such that it is operable in eight directions: upwards, downwards, towards the left, towards the right, diagonally up left, diagonally up right, diagonally down left and diagonally down right. As an alternative, the point on the map to be selected may also be controlled by the operator by means of a computer mouse or a touch pad.

As soon as the point in the map to be selected for the cooking process is reached, a confirmation key can be operated for defining and implementing the selected program point. This key may for instance be provided as a separately movable central key in the control panel or as a vertical shift of the joystick. In the case of a mouse, the input confirmation is generated by a corresponding button action, e.g. by a double click of the left or right mouse button. This analogously applies to the touch pad.

According to the present invention, a control panel for a cooking device, in particular a steam cooking device, is provided comprising an input device for setting a plurality of cooking parameters and a display member which at least displays the cooking parameters and cooking program steps set. Advantageously, the display member comprises an at least two-dimensional map, on which at least two cooking parameters are defined and on which a point in the map may be controlled or selected by action of the operator on the input means.

In an especially preferred manner, the map is a substantially planar surface.

In a further preferred manner, the map is formed as a rectangular monitor surface.

The present invention finally also comprises a cooking device, in particular a steam cooking device, which is characterized in that it comprises a control panel as described above.

Further details, features and advantages of the present invention can be derived from the following description with reference to the drawings.

### BRIEF DESCRIPTION OF THE DRAWINGS

FIG. 1 shows a control panel according to the invention in a front view;

FIG. 2 shows a certain state of the display member according to the invention;

FIG. 3 shows a further state of the display member according to the invention;

FIG. 4 shows the display of a first program step of a cooking process;

FIG. 5 shows the display of a second step in the same cooking process;

FIG. 6 shows the display of a third step in the same cooking process of FIGS. 4 and 5, and

FIG. 7 shows the panels according to the “language of cooking” that correspond to a map including temperature and moisture as cooking parameters.

### DETAILED DESCRIPTION OF PREFERRED EMBODIMENTS

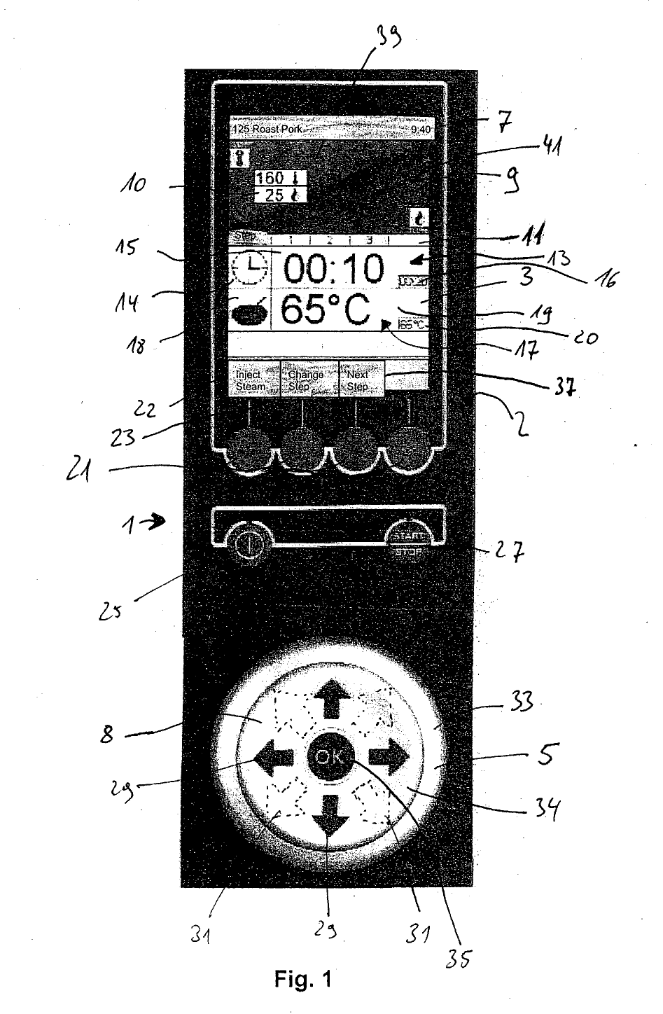

FIG. 1 shows a control panel according to the invention. The control panel comprises a display member 3 and an input device 5.

As can be seen from FIG. 1, the display member 3 consists of a plurality of display panels. According to the embodiment shown, a first display panel 7 is provided in which the type of the food to be cooked is shown. In the example, this is “roast pork”. Below that a map 9 according to the invention is located, which will be described further below. Below that a second display panel 11 is located, in which the number of program steps is given. In FIG. 1, the number of steps shown is 3. The respective method step is then color-illuminated so that the operator immediately recognizes in which method step he is.

Below that a third display panel 13 is located which is divided into three panels: a first left display panel 14 carrying the symbol of a clock, a first main display panel 15 with a time (in FIG. 1 00.10=10 minutes) and a first target value display panel 16 in which the total time of the cooking program step is indicated. In the example shown in FIG. 1, the operator immediately recognizes that 10 minutes of the 20 minute cooking process have already lapsed.

This is followed below by a fourth display panel 17 structured analogously to third display panel 13. In fourth display panel 17, the core temperature is shown, which is symbolically shown in the second left display panel 18. The actual temperature is indicated in the second main display panel 19, and the temperature to be reached is indicated in the second target value display panel 20. In the embodiment shown according to FIG. 1, it can be derived that the target value core temperature of 65° C. has been reached 10 minutes ago and 10 further minutes are required to fully perform the pre-adjusted cooking process.

In addition to the numeric indication in the main display panels 15 and 19, these panels can also be underlined by an analog display, e.g. in the form of a gray bar, which grows towards the right with the progressive duration or increase of the temperature. The operator quickly recognizes the analog target-actual value comparison.

Four operating keys 21 are located below the display member 3 according to the embodiment of FIG. 1. According to a preferred embodiment of the present invention, these four operating keys 21 are formed as capacitive keys. That means that these keys are arranged behind an outer surface 2 of the control panel 1, which can for instance be made of glass. By touching the outer surface 2 in the area of one of the operating keys 21, this key is operated without the operator having to exert pressure onto this key. The special advantage of this embodiment is that the control panel 1 has a closed outer surface 2 which offers an appropriate protection against the electronics arranged therebehind. It is known that the degree of pollution is enormous in kitchens so that the appliances located in the kitchen must be particularly protected against outer influences. This protection is excellently realized by the control panel according to the present invention.

Moreover, the outer surface 2 can also be cleaned easily which is especially favorable in that excellent hygiene is achieved as a special advantage.

By means of one of the operating keys 21, different program control processes and program changing processes can be carried out. The operating keys are advantageously freely selectable. Thus, a respective steam shot, as shown in fifth display panel 22, is generated by the operating key 21 located on the very left that is connected to fifth display panel 22.

The operating key 21 following on the right-hand side enables a program change, as can be seen in FIG. 1 from the indication “change step” in sixth display panel 23. The next operating key 21 follows on the right-hand side. When operating this key, the next step of the cooking process program can be initiated. The operating key arranged on the very right-hand side is not occupied in the example shown. Due to the free programming of the operating keys 21, variable menus can be realized.

As may be seen from FIG. 1, a further key panel with a left and a right key 25 and 27 is located below the operating keys 21. The cooking device is switched on by the left key 25 and the cooking program is started, ended or interrupted by the right key 27.

The operating keys 21 and/or the left and/or right key 25, 27 may comprise light emitting diodes which fulfill corresponding indication functions. Such a LED display is for instance advantageous in the left key 25, since it is indicated to the operator that the apparatus is still in operation and is switched off after operating this key again.

As may be seen from FIG. 1, the input device 5 is arranged in the lower portion of the control panel 1. In the embodiment shown, the input device 5 has the form of a rocker switch 8. Such a rocker switch 8 is known per se. In the embodiment show, it can be operated in eight directions according to the full arrows 29 and dotted arrows 31. The input device has an outer circumferential wall 33 which extend circularly around the operating rocker surface 34.

A central operating key 35 (inscription “OK”) is arranged in the center on the operating rocker surface 34. By pressing this key, the point controlled and selected in the map is implemented into the cooking program.

By means of the input device 5, the climate parameters temperature and humidity in the cooking chamber are determined. In FIG. 1 this point in the map 9 is characterized by the display 10 in which according to the embodiment of FIG. 1 the temperature indication 160 plus thermometer pictogram and 25 plus water drops are indicated. In other words, the cooking process step that took place there was adjusted to 160° C. and to 25% humidity.

By pressing the operating rocker switch 8 onto one of the arrows 29 or 31 the point in the map indicated by the display 10 is shifted accordingly. The incrementation of the map may for instance be 1° C. and 1% humidity. Finer or coarser gradations can also be set. When the choice of the next point has taken place, this point is defined as the next program point by pressing the confirmation key 35 after previously the operating key 21 assigned to the seventh display panel 37 (“next step”) was pressed.

As may also be seen from FIG. 1, the map 9 is basically formed of two partial surfaces which are each formed as a right-angled triangle. The upper partial surface 39 is colored and may for instance be shown in red color, which is the symbol for fire, wherein, as the person skilled in the art may easily recognize, the map 9 is structured such that on the vertical coordinate towards the top temperature is indicated and on the horizontal coordinate the humidity is indicated. The lower partial surface 41 appears for instance in blue color, as a symbol for water, wherein due to the increase of the blue surface from left to right this may be interpreted by the operator as an increase of humidity in the cooking chamber.

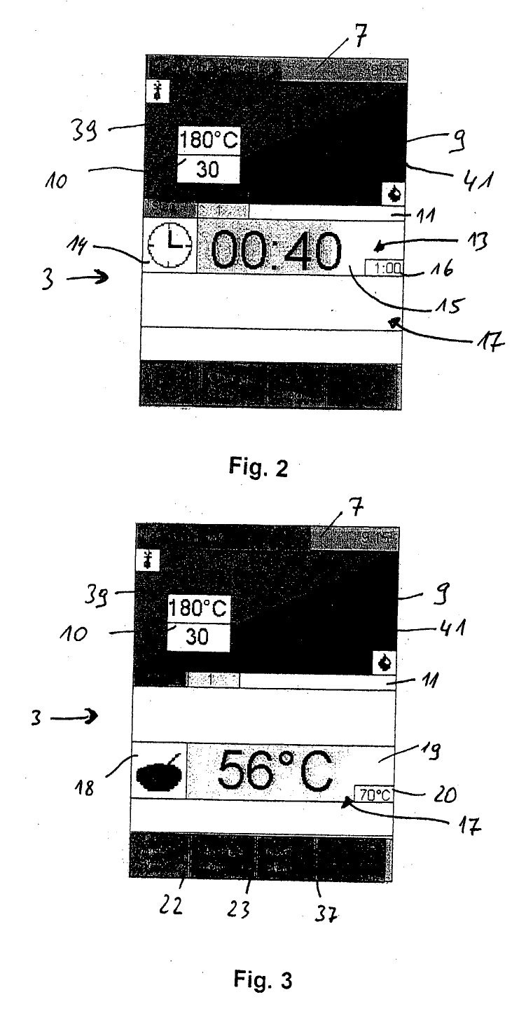

FIG. 2 shows the display member 3 for the cooking process of “Apple Strudel”. The cooking chamber climate was set to 180° C. at 30% humidity. The cooking process is in step 1, wherein this step shall take a total of one hour according to the display panel 16. 40 minutes have already lapsed according to the display panel 15. Since the core temperature of the apple strudel is not determined, the lower display panel 17 is empty.

FIG. 3 shows an alternative cooking process step 1. The cooking chamber climate was again set to 180° C. at 30% humidity, wherein the cooking process shall be controlled according to the core temperature according to the display panel 17. In the example of FIG. 3, the desired temperature of 70° C. according to the display panel 20 is not reached yet. The display panel 19 only shows 56° C.

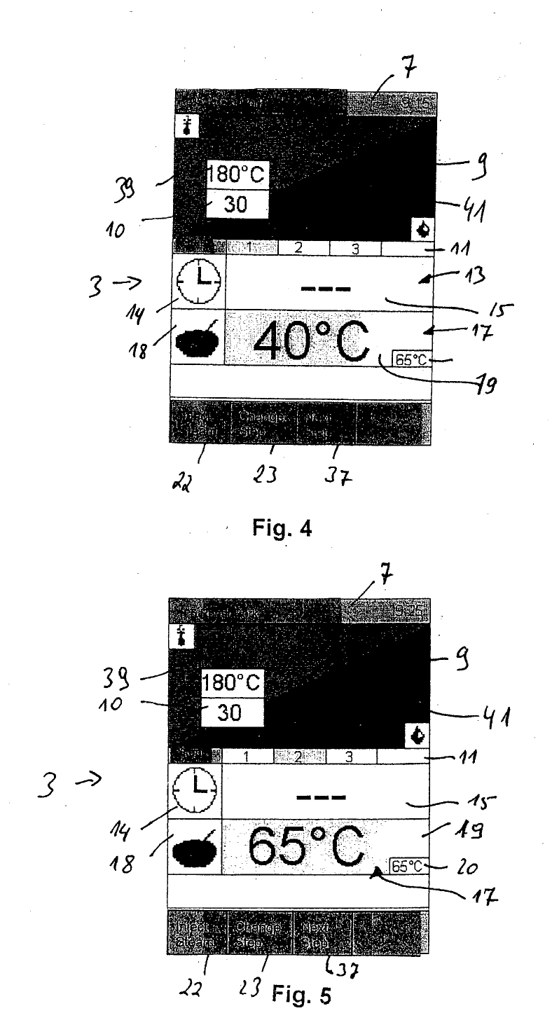

In FIGS. 4 to 6 the cooking process steps 1 to 3 of a cooking process referring to “roast pork” are shown. In step 1, the food is first of all heated at a cooking chamber climate of 180° C. and 30% humidity until a core temperature of 65° C. is reached. According to the display panel 19, only 40° C. has been reached.

FIG. 5 shows a second cooking process step with an unchanged cooking chamber climate compared to the setting of FIG. 4, wherein the actual core temperature of 65° C. is displayed.

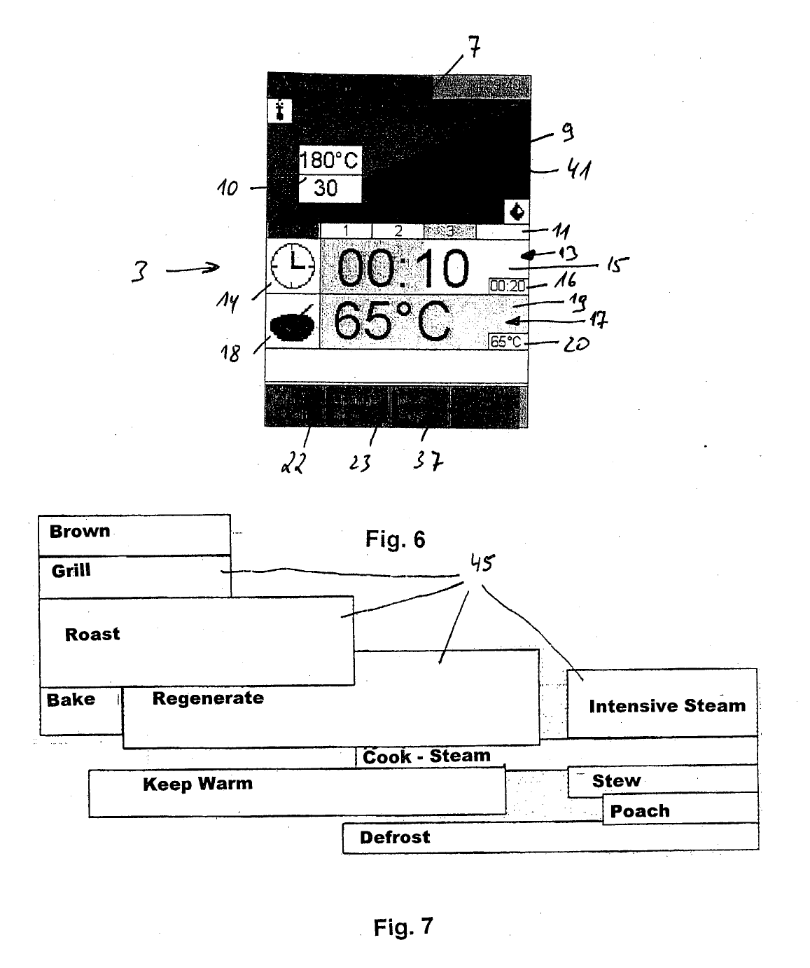

According to FIG. 6 the actual core temperature is indicated in this cooking process in a step 3 at an unchanged cooking chamber climate. This temperature is predefined for 20 minutes according to the target value display panel 16, wherein 10 minutes of cooking time remain. FIG. 6 thus shows a time step following the heating-up step of FIG. 5, as it occurs in multi-stage cooking programs.

FIG. 7 shows in panels 45 different preparation modes for food. Each panel 45 contains a respective step of preparation, wherein the arrangement of the panels with respect to each other is made in that in the map towards the top the temperature and towards the right the humidity are increasingly shown. It results therefrom that the hottest operating mode at a simultaneous lowest humidity “browning” is arranged on the top left. Vice versa is the treatment mode “poaching” arranged at low temperature but very high humidity. When starting a point in map 9 in the outlined panel 45, this panel is indicated and the operator familiar with the “language of cooking” can therefore correctly and very quickly decide whether the started point shall be selected or not.

Thus, a method of treating and preparing food in a cooking device is provided by the present invention, in which cooking parameters can easily be set by the operator. By means of the rocker switch 8, the operator is able to shift the cooking parameter point to be set (display 10) similar to a cursor in the map 9. If the operator presses onto arrows 29 upwards or downwards, the cursor point can also be moved into display panels 13 and 17. By pressing onto the arrows towards the left or right, the corresponding value can be reduced or increased in the respective display panel.

The present method also includes treatment steps and modes such as hot or cold smoking.

Thus, an extremely simple and easily operable display panel is provided by the present invention, wherein the expression “control panel” also comprises the corresponding control means and connection means between input device 5 and display member 3.

A cooking device, which comprises such a control panel 1, can therefore excellently be handled by the operator, wherein an extreme simplification in the input of the individual cooking processes is obtained.

The present invention is not restricted to the embodiments shown. The input device can also be formed as a joystick, wherein advantageously this joystick is arranged on a substantially horizontal or transversely arranged surface element. A computer mouse or a touch pad can also be used as multi-dimensional input devices if they are arranged appropriately.

The map may also be formed three-dimensionally, wherein a third parameter is defined in the third dimension.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of treating and preparing food in a cooking device where a plurality of cooking parameters can be selected by an operator of the cooking device and where the cooking process is carried out through use of an adjustable cooking program, the method comprising:
choosing a point on an at least two-dimensional map through use of an input device wherein at least two cooking parameters for the cooking device are selected, and
implementing the selection into the cooking program as part of the cooking process.

2. The method of claim 1 wherein the temperature, the humidity in the cooking chamber, the velocity of the fan wheel of the cooking device, the browning degree of the food to be cooked, the core temperature of the food to be cooked may be selected as cooking parameters.

3. The method of claim 1 wherein panels are predefined for different cooking processes and are indicated on the map so that the panels can be selected by the input device.

4. The method of claim 1 wherein each point on the map can be reached so that any possible combination of temperature and humidity in the cooking chamber can be selected.

5. The method of claim 1 wherein the input device is a multi-dimensional input device.

6. The method of claim 5 wherein the multi-dimensional input device is a rocker switch, a joystick, a computer mouse or a touch pad.

7. The method of claim 1 wherein a confirmation key is operated to define and implement the selected program point.

8. A control panel for a cooking device comprising:
an input device for setting a plurality of cooking parameters; and
a display member for indicating at least the adjusted cooking parameters and cooking program steps, wherein the display member comprises an at least two-dimensional map on which at least two cooking parameters are defined and on which a point can be controlled by operation of the input device.

9. The control panel of claim 8 wherein the map is a substantially planar surface.

10. The control panel of claim 8 wherein the map is formed as a rectangular monitor surface.

11. The control panel of claim 8 wherein each point on the map can be reached so that any possible combination of temperature and humidity in the cooking chamber can be selected.

12. The control panel of claim 8 wherein the input device comprises a multidimensional input device.

13. The control panel of claim 12 wherein the multi-dimensional input device is a rocker switch, a joystick, a computer mouse or a touch pad.

14. The control panel of claim 12 wherein the multi-dimensional input device further comprises a confirmation key.

15. The control panel of claim 8 wherein the cooking device is a steam cooking device.

16. A device for the treatment and preparation of food comprising a control panel, the control panel having an input device for setting a plurality of cooking parameters and a display member for indicating at least the adjusted cooking parameters and cooking program steps, the display member having an at least two-dimensional map on which at least two cooking parameters are defined and on which a point can be selected by operation of the input device.

17. The device of claim 16 wherein the map is a substantially planar surface.

18. The device of claim 16 wherein the map is formed as a rectangular monitor surface.

19. The device of claim 16 wherein each point on the map can be reached so that any possible combination of temperature and humidity in the cooking chamber can be selected.
